(19) United States
(12) Patent Application Publication
(10) Pub. No.: US 2007/0129683 A1
(43) Pub. Date: Jun. 7, 2007

(54) Fluid detection needle assembly

(76) Inventor: Mark J. Brungardt, Westminister, CO (US)

Correspondence Address: RICK MARTIN;PATENT LAW OFFICES OF RICK MARTIN, PC, 416 COFFMAN STREET, LONGMONT, CO, 80501, US

(21) Appl. No.: 11/296,065

(22) Filed: Dec. 7, 2005

(51) Int. Cl. A61M 5/178

(52) U.S. Cl. 604/168.01; 600/576

(57) ABSTRACT

A fluid detection needle assembly utilized to withdraw a blood sample from an arm of a human being through a needle member. The fluid detection needle assembly replaces a conventional needle assembly having 1) an inlet needle cover member; 2) a detection needle support assembly connected to the needle member; and 3) a fluid receiving tube assembly to receive a human's blood sample therein. The fluid detection needle assembly includes 1) an inlet needle assembly having the needle member; 2) a fluid observation assembly to receive a blood sample from the inlet needle assembly; 3) an intermediate connector assembly; and 4) a discharge needle assembly connected to the intermediate connector assembly. The fluid observation assembly includes a fluid observation section constructed of a clear material operable to receive an initial portion of a human's blood sample to indicate that the needle member has penetrated a blood vessel or vein in the human's arm from which a blood sample is being taken.

### PRIOR ART

A preliminary search was conducted by the applicant herein in the area of structures used to take a fluid sample which is normally used for taking a blood sample from an arm member of a human being.

In the normal course of taking a blood sample from a human being, a needle assembly is used whereupon a needle member is inserted through an outer skin on the arm member of the human being in an effort to penetrate a blood vessel, artery, or vein, hereinafter referred to as blood vein, in subject arm member. In many cases, the process of penetrating a blood vein is not achieved and, therefore, the needle member must then be removed and a second and, perhaps, other attempts must be made to penetrate a blood vein. The fact that the blood vein has not been penetrated is not known to the doctor or nurse attempting to take this blood sample until a collection tube known in a prior art needle assembly is used in order to ascertain whether a successful blood sample will be obtained.

The fluid detection needle assembly of this invention is operable so that a fluid observation assembly clearly shows whether a blood vein has been penetrated and a successful withdrawl of the blood sample will be obtained.

No prior art patents were found in the applicant's search that operate in a manner similar to the applicant's invention herein. This search was conducted on the internet by searching the United States Patent and Trademark Office website (www.uspto.gov).

### BACKGROUND OF THE INVENTION

There has been a need in the area of taking fluid samples, namely blood samples, from human beings in that it would be determined that an inlet needle assembly that is inserted into a human being's arm member has actually penetrated a blood vein and to determine this fact before a collection tube member is attached thereto using vacuum pressure within the subject collection tube member to withdraw a blood sample from the human being's arm member.

The past prior art needle assemblies had no means of determining this before applying the vacuum pressure collection tube member and, therefore, the collection tube member would lose its vacuum and have to be discarded. The needle assembly must be removed from the human's arm member and re-inserted through the outer skin in a second, third, or more attempts to assure that a needle member actually has penetrated the blood vein and a successful blood collection process can be achieved.

The invention herein is operable to provide assurance of penetration of the fluid detection needle assembly into the blood vein and then the collection tube member can be attached thereto to successfully receive a blood sample.

The fluid detection needle assembly can be used by veterinarian's to draw blood and other fluid samples from animals. The invention can be used to withdraw fluid samples other than blood wherein an observation of the fluid to be withdrawn is beneficial before use of the vacuum collection tube member.

### PREFERRED EMBODIMENT OF THE INVENTION

In a preferred embodiment of the invention, a fluid detection needle assembly uses elements known in the prior art such as 1) an inlet needle cover member mounted about an inlet needle assembly; 2) a detection needle support assembly to receive and support the fluid detection needle assembly thereon; and 3) a fluid receiving tube assembly operable to be releasably connected to the detection needle support assembly.

The fluid receiving tube assembly is known in the prior art in having a receiver tube member with a tube stopper assembly on an upper end therein and normally provided with vacuum pressure to assist in withdrawing a blood sample from a human's arm member. The fluid receiving tube assembly is well known and presently used in the prior art for obtaining a blood sample from the arm of a human being.

The detection needle support assembly is operable with the fluid receiving tube assembly in a known conventional manner in order to be connected to each other and withdraw a blood sample through prior art needle assemblies in a known and conventional manner and, therefore, is not part of the invention herein.

The invention herein relates to the fluid detection needle assembly including 1) an inlet needle assembly; 2) a fluid observation assembly connected to the inlet needle assembly; 3) an intermediate connector assembly connected to the fluid observation assembly; and 4) a discharge needle assembly connected to the intermediate connector assembly and operable to transfer a blood sample into the fluid receiving tube assembly.

The inlet needle assembly includes a conventional needle member having 1) an inlet end section; 2) a fluid conveyance tube section integral with the inlet end section; and 3) a connector end section integral with the fluid conveyance tube section.

The inlet end section is provided with a tapered pointed penetration portion on the outer end thereof which is operable to penetrate skin on a human's arm member and into a blood vein while taking a blood sample.

The fluid observation assembly includes 1) a needle support section operable to receive and support the connector end section on the inlet needle assembly; 2) a fluid observation section integral with the needle support section and having a fluid receiving cavity or hole therein; and 3) a connector end section.

The fluid observation section, and mainly the fluid receiving cavity or hole, is operable to receive an initial or first portion of a blood sample therein.

The fluid observation assembly is constructed of a clear material, such as glass or plastic, so that the fluid to be transferred therethough is readily observable from the fluid observation section to determine whether a fluid sample is to be obtained from the present insertion of the inlet needle assembly in a blood vein in a human's arm member or other location.

The intermediate connector assembly includes 1) an observation tube connector section; 2) a flange member integral with the observation tube connector section; 3) a screw anchor section integral with the flange member having external threads thereon for interconnection to the detection needle support assembly; and 4) a discharge needle connector section integral with the screw anchor section and operable to receive and support a discharge needle assembly thereon.

The discharge needle assembly includes 1) a discharge needle member connected to the discharge needle connector section; and 2) a discharge needle cover assembly connected to the discharge needle connector section operable to enclose and seal about the discharge needle member.

The discharge needle member is similar to the inlet needle assembly having 1) a discharge connector end section connected to the discharge needle connector section of the intermediate connector assembly; 2) a discharge conveyance tube section integral at one end with the discharge connector end section; and 3) a discharge end section integral with an outer end of the discharge conveyance tube section.

The discharge needle cover assembly is constructed of a flexible rubber material and having a tapered covered end penetration portion.

The discharge needle cover assembly has 1) a cover connector section connected to the discharge needle connector section on the intermediate connector assembly section; 2) a needle protection section of cylindrical shape integral with the cover connector section; and 3) an end cover section integral with the needle protection section and providing a sealing cover over the discharge needle member.

The discharge needle cover assembly is operable to keep the discharge needle member in a sealed sterile condition before having the end cover section penetrated by the discharge needle member on allowing a blood sample to flow therethrough and to be placed in a fluid receiving tube assembly in a conventional manner.

A second embodiment of this invention is provided with a fluid detection needle apparatus which is identical to the first embodiment except the fluid observation assembly is provided with a fluid receiving hole therein instead of a fluid receiving cavity, and being the same size as the needle support hole in the needle support section of the first embodiment. This is easier in manufacturing and less costly to do so and reduces the size of a fluid blood sample to be observed through the fluid observation assembly.

In another embodiment of this invention, a fluid detection needle mechanism is provided and, instead of the fluid receiving cavity or hole therein, is provided with a fluid receiving globe or ball to provide more volume therein for easy observation of an initial blood sample being withdrawn from a human's arm member or other location.

### OBJECTS OF THE INVENTION

One object of this invention is to provide a fluid detection needle assembly which will replace a conventional needle assembly used to withdraw fluid, and normally a blood sample, from an arm member of a human being.

Another object of this invention is to provide a fluid detection needle assembly utilized with a conventional 1) inlet needle cover member; 2) a needle support assembly; and 3) a fluid receiving tube assembly with the fluid detection needle assembly having a fluid observation assembly therewith whereupon a fluid or blood sample can be observed thus indicating that a successful penetration by an inlet needle assembly of a blood vein on the arm of a human from which a blood sample is to be obtained.

One other object of this invention is to provide a fluid detection needle assembly having an inlet needle assembly connected to a fluid observation assembly which, in turn, is connected to an intermediate connector assembly; and a discharge needle assembly is connected to the intermediate connector assembly.

A further object of this invention is to provide a fluid detection needle assembly having thereon a fluid observation assembly whereupon an initial drawing of a blood sample is visibly observed through the fluid observation assembly to indicate that the inlet needle assembly has properly penetrated a human's blood vein so that, at that time, it is known that a successful withdrawing of the blood sample will be obtained as the inlet needle assembly has been properly inserted.

One further object of this invention is to provide a fluid detection needle assembly including 1) an inlet needle assembly; 2) a fluid observation assembly attached to one end of the inlet needle assembly; 3) an intermediate connector assembly connected to another outer end of the fluid observation assembly; and 4) a discharge needle assembly connected to the intermediate connector assembly and having a discharge needle member enclosed and sealed by a discharge needle cover assembly.

Another further object of this invention is to provide a fluid detection needle assembly having an inlet needle assembly operable in a conventional manner to obtain a blood sample, and having a fluid observation assembly whereupon an initial withdrawing of blood to obtain subject blood sample is operable to provide a fluid observation section to ascertain whether a successful penetration of a human's arm and into a blood vein has been achieved by the inlet needle assembly before attaching a fluid receiving tube assembly thereto, whereupon failure to provide a successful penetration of the human's blood vein, the fluid receiving tube assembly is inoperative because vacuum pressure therein will be lost due to not having obtained proper penetration of the needle assembly in subject blood vein.

Still, one other object of this invention is to provide a fluid detection needle assembly that is sturdy in construction; new and novel in operation; easy to perform maintenance operations thereon; and substantially maintenance free.

Various other objects, advantages and features of the invention will become apparent to those skilled in the art from the following discussion taken in conjunction with the accompanying drawings in which:

### FIGURES OF THE INVENTION

FIG. 1 is an exploded perspective view of the fluid detection needle assembly of this invention utilized with other known elements needed to obtain a fluid sample;

FIG. 2 is a sectional view of the fluid detection needle assembly taken along line 2-2 in FIG. 1 with some known elements therewith;

FIG. 3 is a side elevational view of this invention being the fluid detection needle assembly;

FIG. 4 is an exploded perspective view of the fluid detection needle assembly of this invention;

FIG. 5 is a view illustrating the operation of the fluid detection needle assembly of this invention on withdrawing an initial portion of a blood sample from a blood vein in a human's arm; and

FIG. 6 is a view similar to FIG. 5 illustrating the successful withdrawing of a blood sample from a blood vein in the human's arm through the fluid detection needle assembly of this invention into a fluid receiving tube assembly or blood sample collection tube member.

The following is a discussion and description of a preferred specific embodiment of the new fluid detection needle assembly of this invention, such being made with reference to the drawings, whereupon the same reference numerals are used to indicate the same or similar part and/or structure. It is to be understood that such discussion and description are not to limit the scope of the invention.

### DESCRIPTION OF THE INVENTION

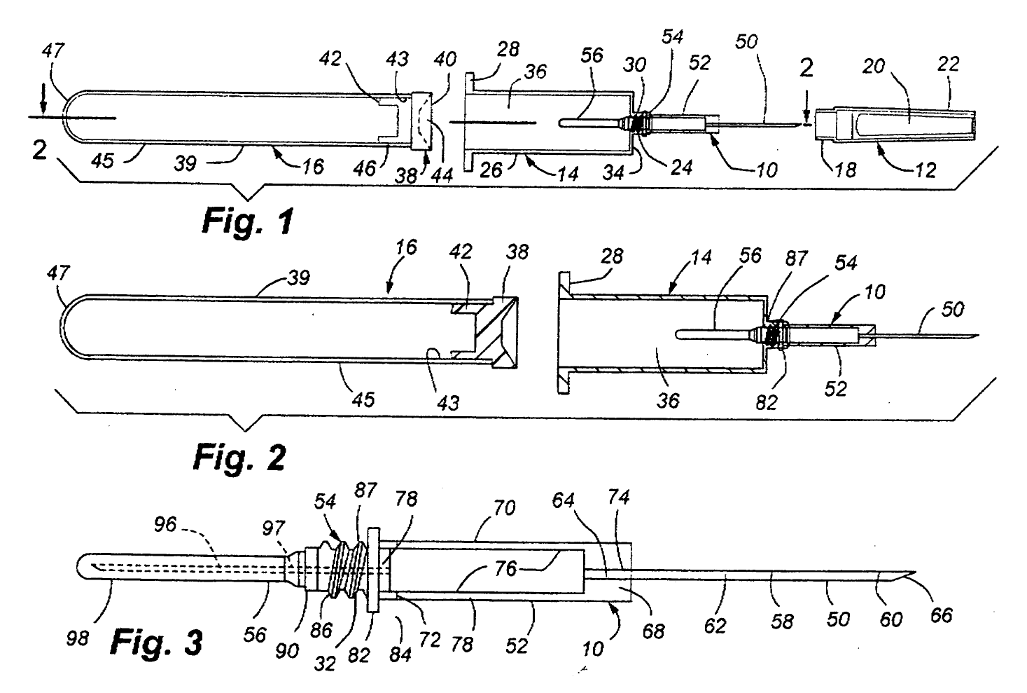

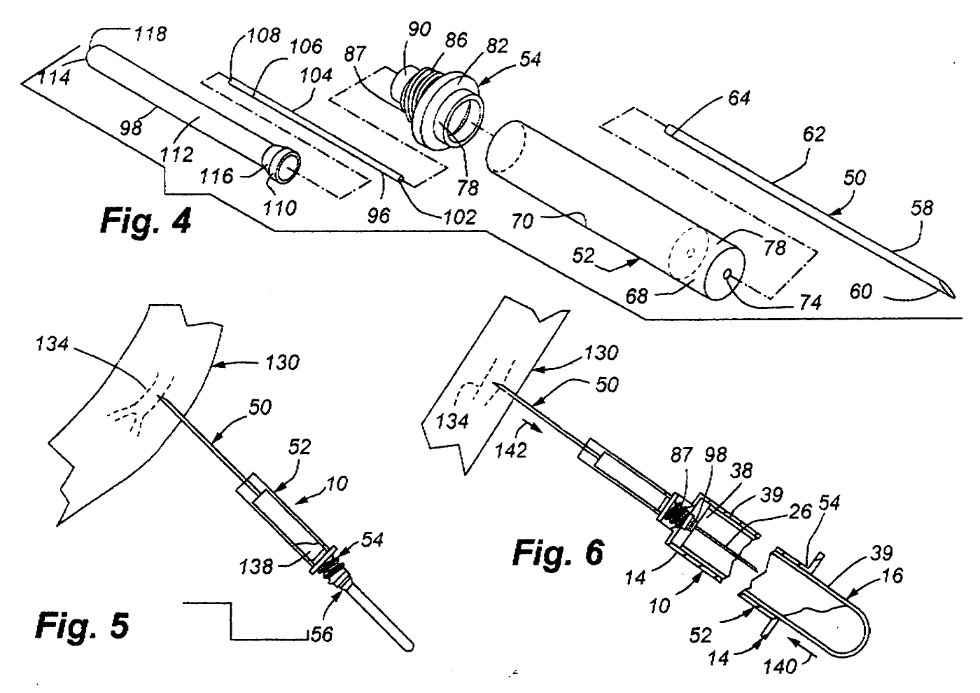

On referring to the drawings in detail and, in particular to FIG. 1, a fluid detection needle assembly of this invention, indicated generally at 10, is shown with other elements that are known in the prior art to be utilized with a prior art needle assembly in order to obtain a blood sample from, for example, an arm of a human being in a conventional manner.

The fluid detection needle assembly 10 is operable, and its' novelty achieved, in detecting an initial portion of a blood sample to indicate that a successful penetration of a blood vein in a human's arm member has been achieved.

On referring to FIG. 1, the fluid detection needle assembly 10 of this invention is utilized with 1) an inlet needle cover member 12 to provide an enclosure and sanitary protection of a needle member on a conventional or the fluid detection needle assembly 10 of this invention; 2) a detection needle support assembly 14 operable to receive and support the fluid detection needle assembly 10 as will be explained; and 3) a fluid receiving tube assembly 16 operable to receive and collect a blood sample therein.

The inlet needle cover member 12 is of a conventional known structure having 1) an attachment connector section 18 integral with a needle protection cavity 20; and 2) an outer support or grasp surface 22 to receive a human's fingers thereon for pulling to remove from the detection needle support assembly 14

The detection needle support assembly 14 includes 1) a connector end assembly 24 to receive and support a portion of the fluid detection needle assembly 10 therein; 2) a receiving tube connector section 26 to support and receive the fluid receiving tube assembly 16 therein as will be noted; and 3) a guide and grasping flange member 28.

The connector end assembly 24 includes a connector hub 30 having anchor threads 32 thereon and an end wall 34.

The receiving tube connector section 26 is of a hollow cylindrical shape having an internal tube cavity 36 to receive the fluid receiving tube assembly 16 therein as will be explained.

The guide flange member 28 operates in a known manner to receive a human's fingers thereagainst similar that found on a prior art syringe assembly.

The fluid receiving tube assembly 16 includes 1) a tube stopper assembly 38; and 2) a receiver tube member 39 of a clear glass or plastic material having the tube stopper assembly 38 mounted in an open upper end thereof.

The tube stopper assembly 38 includes 1) an outer sealing end section 40; and 2) a stopper tube section 42 operable to engage and seal with an inner surface of an upper portion of the receiver tube member 39 as will be noted.

The outer sealing end section 40 is provided with a needle indentation portion 44 to receive and be penetrated by a discharge needle member of the fluid detection needle assembly 10 as will be explained.

The stopper tube section 42 is provided with an outer side wall portion 46 operable to be sealingly engagable with an outer open end portion of the receiver tube member 39 in a known manner.

The receiver tube member 39 is provided with an outer entrance opening 43 integral with a tube body section 45 and an outermost end wall section 47.

The receiver tube member 39 is a known test tube type structure manufactured and processed with a vacuum pressure therein, whereupon, when the tube stopper assembly 38 is penetrated by the discharge needle member, the vacuum pressure therein will aid in obtaining and pulling a blood sample from a human's arm member in a known conventional manner.

The receiver tube member 39 is constructed of a clear material, such as glass or plastic, so that the blood sample being placed therein is readily observed.

As noted in FIG. 2, the fluid detection needle assembly 10 includes 1) an inlet needle assembly 50 of a conventional nature; 2) a fluid observation assembly 52 having one end connected to a discharge end of the inlet needle assembly 50; 3) an intermediate connector assembly 54 operable to be engagable and connected to the detection needle support assembly 14; and 4) a discharge needle assembly 56 interconnected to the intermediate connector assembly 54.

The inlet needle assembly 50 includes a needle member 58 having an inlet end section 60 integral with a fluid conveyance tube section 62 which, in turn, is integral with a connector end section 64.

The inlet end section 60 is provided with a tapered penetration portion 66 operable to initially penetrate the skin on a human's arm or other location from which a blood sample is to be taken.

As noted in FIG. 3, the fluid observation assembly 52 is constructed of a clear material, such as plastic or glass, and having 1) a needle support section 68 to receive and support the needle member 58 therein; 2) a fluid observation section 70 into which an initial portion of a blood sample is placed; and 3) a connector end section 72 operable to be attached to the intermediate connector assembly 54 as will be explained.

The needle support section 68 is provided with a needle support hole 74 to receive and support the connector end section 64 of the needle member 58 therein.

The fluid observation section 70 is provided with an enlarged fluid receiving cavity or hole 76 therein to receive an initial portion of a blood sample. A portion of the inner surface of the fluid receiving cavity or hole 76 may be provided with a white coating on a portion thereof so that the initial blood sample received therein can be readily observed and contrasted with the white coating.

The intermediate connector assembly 54 has 1) an observation tube connector or hub 78 to receive and support the connector end section 72 of the fluid observation assembly 52 thereon; 2) a flange member 82 for ease of rotation of the entire intermediate connector assembly 54; 3) a screw anchor section 86 having external threads 87 thereon to be mounted within and connected to the detection needle support assembly 14; and 4) a discharge needle connector section 90 to receive and support the discharge needle assembly 56 thereon as will be explained.

The discharge needle assembly 56 includes 1) a discharge needle member 96 secured to the discharge needle connector section 90 and mounted in a support hole 97 therein; and 2) a discharge needle cover assembly 98 constructed of a flexible rubber or plastic material for reasons to be explained.

The discharge needle member 96 includes 1) a discharge connector end section 102 mounted within and secured to the discharge needle connector section 90 of the intermediate connector assembly 54; 2) a discharge conveyance tube section 104 integral with the discharge connector end section 102; and 3) a discharge end section 106 integral with an outer end of the discharge conveyance tube section 104.

The discharge end section 106 is provided with a tapered covered penetration portion 108 operable to penetrate a portion of the discharge needle cover assembly 98 when obtaining and collecting a blood sample to be placed within the fluid receiving tube assembly 16 as will be explained.

The discharge needle cover assembly 98 is operable to sealingly receive and cover the needle discharge member 96.

The discharge needle cover assembly 98 includes 1) a cover connector section 110; 2) a main needle protection section 112 integral with an outer end of the cover connector section 110; and 3) an end cover section 114 to sealingly enclose and protect an outer end of the discharge needle member 96, namely, the penetration portion 108.

The cover connector section 110 is provided with a stepped portion 116 operable to be sealingly connected to the discharge needle connector section 90.

The end cover section 114 is provided with an outer penetration portion 118 to be penetrated by the tapered cover penetration portion 108 of the discharge needle member 96 when taking a blood sample in a manner to be explained.

A second embodiment of the invention, being a fluid detection needle apparatus, may be provided whereupon the fluid receiving cavity or hole 76 in the first embodiment has been replaced with a fluid receiving hole of the same diameter as the needle member 58 and the discharge needle member 96 being of less volume than the first embodiment and less costly to produce in manufacturing cost.

It is to be noted that the fluid receiving cavity or hole 76 could be of various sizes and shapes and even of a globular or circular cavity to vary the amount of an initial portion of the blood sample to be received therein. The size of this fluid receiving cavity or hole 76 may be regulated as desired, depending on the fluid to be placed therein as there has to be some compression of air therein to allow the initial portion of the fluid sample to be received therein without flowing through the discharge needle member 96 or causing a penetration or rupture of the discharge needle cover assembly 98.

### Use and Operation of the Invention

In the use and operation of this invention, the fluid detection needle assembly 10 is operable to utilize other elements therewith, namely in the process of drawing a blood sample from a human's arm 130 or other body location.

The main novelty of this invention is providing a new and novel fluid detection needle assembly 10 having a fluid observation assembly 52 whereupon an initial first portion of a blood sample to be withdrawn is observed through the fluid observation assembly 52 which is constructed of a clear glass or plastic material. It is a means to positively ascertain that the inlet needle assembly 50 and the main inlet end section 60 with the tapered penetration portion 66, has been placed within the confines of a human's or animal's blood vein 134.

The fluid detection needle assembly 10 is initially provided with the inlet needle cover member 12 placed over and about and protecting the inlet needle assembly 50 and having the attachment connector section 18 firmly engaged about the connector end section 72 of the fluid observation assembly 54 and being abutted against an outer flat surface of the flange member 82.

As noted in FIG. 1, the first step after removing the inlet needle cover member 12 is to reveal the needle member 58 whereupon, after preparation of the human's arm 130 with a tourniquet, a rubber strap, or the like, and a cleansing antiseptic in order to proceed with taking a blood sample, the tapered penetration portion 66 is inserted through a human's outer skin, normally in a forearm portion of the human's arm 130, in an attempt to place the end thereof in a human's blood vein 134.

On proper placement of the tapered penetration portion 66 in the blood vein 134, the blood pressure in the human body would act to transfer blood therefrom through the needle member 58 into the fluid observation assembly 52 and, more particularly, the fluid receiving cavity or hole 76. Due to the clear nature of the glass or plastic fluid observation assembly 52, it would be visible as a fluid 138 therein (FIG. 5). As previously discussed, an inner surface of the fluid receiving cavity or hole 76, being painted white, as noted at 138, would readily allow observation of the red blood sample therein. This would indicate that the blood vein 134 has been properly penetrated and a full blood sample can be taken.

The next step would be to attach the detection needle support assembly 14 which can then be screwed on the thread members 87 to achieve the interconnection therebetween as noted in FIGS. 2 and 6.

The next step, as noted in FIG. 6, would be to take the fluid receiving tube assembly 16 and move the same within and axially of the needle detection support assembly 14. A lateral movement is shown by an arrow member 140.

Next, the tube stopper assembly 38, being of a rubber material, would then contact the discharge cover assembly 98 which encloses the discharge needle member 96. Continued movement, as shown by the arrow 140, will then cause the rubber discharge needle cover assembly 98 to be penetrated by the tapered cover penetration portion 108 of the discharge end section 106 of the discharge needle member 96.

The vacuum pressure in the receiver tube member 39 acts to move a blood sample 145 into the tube body section 45 as noted in FIG. 5.

As the receiver tube member 39 receives a desired blood sample 145 therein, the fluid receiving tube assembly 16 is removed from the detection needle support assembly 14 and is moved as shown by an arrow 146. The tube stopper assembly 38 is self-sealing on removing the discharge end section 106 of the discharge needle member 96 therefrom.

Next, the fluid detection needle assembly 10 and interconnected detection needle support assembly 14 and, more particularly, the inlet needle assembly 50, is removed from the blood vein 134 in the human's arm 130 in a conventional manner. Then, this unit is considered a used product and disposed of in a sanitary and safe manner to prevent any human contact with a portion of the blood sample 145.

Finally, the blood sample 145 in the receiving tube member 39 is preserved and sent to a laboratory for testing as requested and required.

As the fluid receiving tube assembly 16 has the receiver tube member 39 with a vacuum pressure therein, this vacuum pressure will then pull a human's blood sample through the inlet needle assembly 50, the fluid observation assembly 52, the intermediate connector assembly 54 and the discharge needle member 96 to fill the test tube type receiver tube member 39 with the fluid, in this case a human's blood therein, until a required blood sample 145 has been placed within the receiver tube member 39.

Then, the fluid receiving tube assembly 16 can be readily removed and having the discharge needle member 96 moved outwardly of the tube stopper assembly 38 of the fluid receiving tube assembly 16.

The outer sealing end section 40 of the tube stopper assembly 38 is of a self-sealing nature when the discharge needle member 96 has been removed therefrom.

Next, the fluid detection needle assembly 10 of this invention can be discarded in a known sanitary manner to prevent any human blood from contacting or being placed in an unwanted position.

After the blood sample 145 has been collected in the fluid receiving tube assembly 16 and removed from the detection needle support assembly 14, the fluid detection needle assembly 10 can be then discarded in a known sanitary manner. Also, the inlet needle assembly 50 is removed from the human's arm on movement and direction as shown by an arrow 146 in FIG. 6.

The fluid detection needle assembly 10 of this invention is a relatively small but very important improvement over the conventional needle assemblies presently used in the prior art in order to obtain a blood sample from a human's arm or other chosen locations. The savings of not having to throw away a fluid receiving tube assembly 16 is considerably noting the large amount of blood samples taken yearly on human's for health reasons or when donating blood.

The fluid detection needle assembly 10 is sturdy in construction; reliable in obtaining a blood sample and determining whether a blood vein has been penetrated before removing the blood sample into a collection tube; and substantially maintenance free.

While the invention has been described in conjunction with a preferred specific embodiment thereof, it will be understood that this description is intended to illustrate and not to limit the scope of the invention, which is defined by the following claims:

I claim:

1. A fluid detection needle assembly used to withdraw a blood sample from a medical patient, said needle assembly comprising:
an inlet needle assembly having a needle member with a distal tip operable to penetrate a patient's vein in order to obtain a blood sample therefrom;
a fluid observation assembly connected to said needle member at a proximal end and operable to receive an initial portion of the blood sample therein to indicate that said needle member has penetrated the patient's vein;
wherein said fluid observation assembly has a portion constructed of a transparent material so that the initial portion of the blood sample is readily observed externally of said fluid observation assembly; and
a discharge needle member directly connected to said fluid observation assembly at a distal end of the fluid observation assembly opposite the inlet needle assembly and operable to transfer the blood sample into a fluid receiving tube assembly.

2. (canceled)

3. A fluid detection needle assembly as described in claim 1 including:
a discharge needle cover assembly mounted about said discharge needle member in a sealing manner to prevent any portion of the blood sample from flowing through said discharge needle member unless to be placed intentionally in said fluid receiving tube assembly.

4. A fluid detection needle assembly as described in claim 3, including;
said fluid receiving tube assembly having a tube stopper assembly mounted in a sealing manner in a receiver tube member operable to receive the blood sample therein; and
said tube stopper assembly is moved against an outer end of said discharge needle cover assembly and said discharge needle member penetrates said discharge needle cover assembly and the blood sample is transferred into said receiver tube member.

5. A fluid detection needle assembly as described in claim 4, wherein:
said receiver tube member having a vacuum pressure therein to assist in moving the blood sample from said inlet needle assembly through said fluid observation assembly and said discharge needle member and into said receiver tube member.

6. A fluid detection needle assembly as described in claim 4, including:
a detection needle support assembly and having a receiving tube connector section to receive said receiver tube member therein to guide said tube stopper assembly into engagement with said discharge needle member.

7. A fluid detection needle assembly as described in claim 4, wherein:
said discharge needle cover assembly constructed of a flexible material that can be penetrated by said discharge needle member and moved axially on said discharge needle member in a collapsed manner when transferring the blood sample into said receiver tube member and deposited into said fluid receiving tube assembly.

8. A fluid detection needle assembly operable to take a person's blood sample, said needle assembly comprising:
an inlet needle assembly having a needle member operable to penetrate a person's skin to place an inlet end section in a person's vein to receive the blood sample therefrom; and
said needle member connected to a fluid observation assembly having a clear portion through which an initial portion of the blood sample will be observed to clearly indicate that said inlet end section has penetrated the person's vein and a blood sample will be obtained;
said fluid observation assembly having a fluid observation section constructed of a clear material so that the blood sample can be observed therein;
a discharge needle assembly secured directly by a rigid non tubular assembly to said fluid observation assembly;
said discharge needle assembly includes a discharge needle member enclosed by a discharge needle cover assembly;
whereby said discharge needle cover assembly will maintain the initial blood sample in said discharge needle member before transferring into a fluid receiving tube assembly; and
a detection needle support assembly connected to said intermediate connector assembly and operable to receive and guide a fluid receiving tube assembly into contact with said discharge needle assembly when placing the blood sample into said fluid receiving tube assembly.

9. (canceled)

10. A fluid detection needle assembly as described in claim 8, wherein:
said fluid observation section of a cylindrical shape so that an initial portion of the blood sample will move therein for ease of observation thereof.

11. A fluid detection needle assembly as described in claim 8, including:
a portion of an inner wall of said fluid observation section is painted to obtain a contrast with red blood sample for ease of observation thereof.

12. (canceled)

13. (canceled)

14. A fluid detection needle assembly as described in claim 8, wherein:
said fluid receiving tube assembly includes a tube stopper assembly mounted within and forming a seal with a receiver tube member operable to receive the blood sample therein.

15. A fluid detection needle assembly as described in claim 14, wherein:
said tube stopper assembly constructed of a self-sealing material which, when penetrated by said discharge needle member and withdrawn therefrom, will close and seal the first hole made by said discharge needle member.

16. A fluid detection needle assembly used to withdraw a blood sample from a medical patient, said needle assembly comprising:
a needle member used to penetrate the medical patient's skin and place an inlet end section in a patient's vein in order to remove a blood sample therefrom;
a fluid observation assembly connected to said needle member operable to receive an initial portion of the blood sample therein;
a discharge needle member directly connected to said fluid observation assembly at an opposite end from the needle member used to penetrate the medical patient's skin; and
said discharge needle member being operable to transfer the blood sample into a fluid receiving tube assembly.

17. A fluid detection needle assembly as described in claim 16, including:
a discharge needle cover assembly mounted about said discharge needle assembly; and
said discharge needle cover assembly selectively penetrated by said discharge needle member to place a blood sample in said fluid receiving tube assembly.

18. A fluid detection needle assembly as described in claim 16, including:
an inlet needle cover member operable to be selectively mounted about said needle member and said fluid observation assembly to protect and prevent damage thereto.

19. A fluid detection needle assembly as described in claim 16, including:
a detection needle support assembly connected to said fluid observation assembly and operable to support and guide said fluid receiving tube assembly when placing a blood sample therein.

20. A fluid detection needle assembly as described in claim 19, wherein:
said fluid receiving tube assembly provided with a vacuum pressure therein to assist in moving a blood sample from medical patient's vein into said fluid receiving tub assembly.

21. A blood sample needle assembly comprising:
an inlet needle having a working tip end and a distal end;
a see through rod affixed to the distal end of the inlet needle;
a discharge needle affixed to a distal end of the see through rod opposite the end of the inlet needle; and
wherein a flash of blood is visible in the see through rod upon entry of the working tip into a patient's blood vessel.

22. The needle assembly of claim 21, wherein the see through rod further comprises a support assembly having a receiving tube connector section to receive a receiver tube.
